(19) United States
(12) Patent Application Publication
(10) Pub. No.: US 2026/0335716 A1
(43) Pub. Date: Oct. 29, 2026

(54) SYSTEMS AND METHODS FOR PERSONAL TRAINING

(71) Applicant: HILLMED, INC., Houston, TX (US)

(72) Inventors: Yingchun ZHANG, Katy, TX (US); Nicholas DIAS, Houston, TX (US)

(21) Appl. No.: 19/483,676

(22) PCT Filed: May 13, 2024
(86) PCT No.: PCT/US2024/029120

§ 371 Date: Nov. 12, 2025

Related U.S. Application Data
(60) Provisional application No. 63/465,950, filed on May 12, 2023.
(60) Provisional application No. 63/543,744, filed on Oct. 12, 2023.

(51) Int. Cl. A63B 24/00; A61B 5/00; A61B 5/259; A61B 5/296; A61B 5/397

(52) U.S. Cl. CPC A63B 24/0075; A61B 5/0024; A61B 5/259; A61B 5/296; A61B 5/397; A61B 5/6804; A61B 5/7267; A61B 5/746; A63B 24/0062; A61B 2562/06; A63B 2214/00; A63B 2220/40

(57) ABSTRACT

A system for a personal training includes an electromyography (EMG) sensor, a processor, and a memory coupled to the processor. The memory has instructions stored thereon, which when executed by the processor, cause the system to: receive a first dataset including EMG data and/or accelerometer data from a first user; train a machine learning network using the first dataset to generate a preferred training condition; detect, by the sensor, EMG data and/or accelerometer data from a second user; determine, based on the detected EMG data and/or accelerometer data from the second user, a second dataset corresponding to a training action by the second user; and determine, based on a comparison of the preferred training condition with the second dataset, a recommended training condition for the second user.

### CROSS-REFERENCE TO RELATED APPLICATION AND CLAIM OF PRIORITY

This application claims the benefit of, and priority to, U.S. Provisional Patent Application No. 63/465,950, filed on May 12, 2023 and U.S. Provisional Patent Application No. 63/543,744 filed on Oct. 12, 2023, the entire contents of which are hereby incorporated herein by reference.

### TECHNICAL FIELD

The present application relates to systems and methods for personal training, and, more specifically, to a system and method for personal training, which utilizes artificial intelligence.

### BACKGROUND

Personal training is a popular way to achieve fitness goals, but training alone without proper guidance can lead to several challenges and risks. For example, a user may not be fully aware of the correct form, techniques, and/or best practices for various exercises. This can lead to inefficient workouts or, worse, injury. A common mistake among novice trainers is improper muscular form, (e.g., lower back rounding, knee bending, long stride length), which can cause unnecessary muscle strain that inhibits fitness progress. Moreover, an individual may lack a structured training plan with appropriate progression, or a workout tailored to specific needs, goals, and fitness levels. Overall, proper guidance and feedback are common pitfalls for achieving fitness goals which, when available, can ensure that a user has flexibility, strength, and endurance to prevent imbalances and promote overall fitness.

Accordingly, there is a need for an improved personal training systems that can provide users with real-time feedback on a user's exercise form and technique.

### SUMMARY

In accordance with aspects of the present disclosure, a system for personal training includes a sensor, a processor, and a memory coupled to the processor. The sensor is configured to detect EMG data and/or accelerometer data. The memory has instructions stored thereon, which when executed by the processor, cause the system to: receive a first dataset including EMG data and/or accelerometer data from a first user; train a machine learning network using the first dataset to generate a preferred training condition; detect, by the sensor, EMG data and/or accelerometer data from a second user; determine, based on the detected EMG data and/or accelerometer data from the second user, a second dataset corresponding to a training action by the second user; and determine, based on a comparison of the preferred condition with the second dataset, a recommended training condition for the second user.

In another aspect of the present disclosure, the sensor may include a pod assembly and a conductive substrate.

In yet another aspect of the present disclosure, the pod assembly may include an amplifier, an accelerometer, and/or an analog-to-digital (ADC) converter.

In a further aspect of the present disclosure, the accelerometer may be a tri-axial accelerometer configured to detect acceleration along three axes.

In yet a further aspect of the present disclosure, the conductive substrate may include a conductive sticker configured to attach to the second user's skin.

In another aspect of the present disclosure, the conductive substrate may include a conductive fabric configured to be woven into a garment worn by the second user.

In yet another aspect of the present disclosure, the instructions, when executed by the processor, may further cause the system to output an alert indicating real-time feedback corresponding to the recommended training condition. The alert may include a haptic, visual, and/or auditory cue.

In a further aspect of the present disclosure, the instructions, when executed by the processor, may further cause the system to analyze, by the machine learning network, the second dataset to determine a plurality of features corresponding to the training action by the second user; and preprocess the EMG data and/or accelerometer data from the second user prior to determining the second dataset. The plurality of features may include an EMG amplitude, an amplitude distribution, an inter-muscular coherence, an intra-muscular connectivity, and/or a weighted phase lag coherence. Preprocessing the EMG data and/or accelerometer data may include removing noise, normalizing data, and/or segmenting data.

In yet a further aspect of the present disclosure, the recommended training condition may correspond to a medical application, a sports application, and/or a recreational application.

In another aspect of the present disclosure, a plurality of sensors may be communicatively coupled using Bluetooth low energy (BLE) to form a network.

In yet another aspect of the present disclosure, the EMG data may correspond to a signal generated by a muscle of the second user.

In accordance with aspects of the present disclosure, a processor-implemented method for personal training includes: receiving a first dataset including at least one of EMG data or accelerometer data from a first user; training a machine learning network using the first dataset to generate a preferred training condition; detecting, by a sensor, at least one of EMG data or accelerometer data from a second user; determining, based on the detected at least one of EMG data or accelerometer data, a second dataset corresponding to a training action by the second user; and determining, based on a comparison of the preferred training condition with the second dataset, a recommended training condition for the second user.

In accordance with aspects of the present disclosure, a non-transitory computer readable storage medium includes instructions that, when executed by a computer, cause the computer to perform a method for personal training, the method comprising: receiving a first dataset including at least one of EMG data or accelerometer data from a first user; training a machine learning network using the first dataset to generate a preferred training condition; detecting, by a sensor, at least one of EMG data or accelerometer data from a second user; determining, based on the detected at least one of EMG data or accelerometer data, a second dataset corresponding to a training action by the second user; and determining, based on a comparison of the preferred training condition with the second dataset, a recommended training condition for the second user.

### BRIEF DESCRIPTION OF THE DRAWINGS

A better understanding of the features and advantages of the disclosed technology will be obtained by reference to the following detailed description that sets forth illustrative embodiments, in which the principles of the technology are utilized, and the accompanying drawings of which:

FIG. 1 is an illustration of a personal training system, in accordance with aspects of the present disclosure;

FIG. 2 is a block diagram of exemplary components of the controller of FIG. 1, in accordance with aspects of the present disclosure;

FIG. 3 is a block diagram of a machine learning network with inputs and outputs of a deep learning neural network, in accordance with aspects of the present disclosure;

FIG. 4 is a diagram of layers of the machine learning network of FIG. 3, in accordance with aspects of the present disclosure;

FIG. 5 is an illustration of a sensor within the system of FIG. 1, in accordance with aspects of the present disclosure; and

FIG. 6 is a flow diagram of an exemplary use of the system of FIG. 1, in accordance with aspects of the present disclosure.

### DETAILED DESCRIPTION

The present application relates to systems and methods for personal training, and, more specifically, to a system and method for personal training, which utilizes artificial intelligence and machine learning.

Many traditional feedback methods allow ample room for various inefficiencies in feedback transmitted as personalized parameters are not consistently and accurately recorded, thereby paving the way for repeated workout faults and increased ineffectiveness. As such, the ability to provide and receive personalized real-time user exercise feedback during one's workout routine has proven to be a valuable part of efficient and injury-free training. The disclosed technology improves the overall effectiveness of an individual's workout routine and mitigates the likelihood of injury.

The personalized exercise guidance system of the present disclosure is designed to provide real-time feedback on a user's exercise form and technique through the integration of wireless sensors, an accelerometer, an AI and/or machine learning model, and a mobile device. The wireless sensors are configured to be attached to the user's body to measure muscle activity, while the accelerometer is configured to monitor the user's movement patterns. These data streams are combined and transmitted to the mobile device via a wireless module, allowing the AI model to assess the user's exercise form and technique. The mobile device is then configured to display the analyzed information and provide real-time feedback to help the user adjust their movements towards the generalized model of proper action.

For the purpose of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Various alterations, rearrangements, substitutions, and modifications of the features illustrated herein, and any additional applications of the principles of the present disclosure as illustrated herein, which would occur to one skilled in the relevant art and having possession of this disclosure, are to be considered within the scope of the present disclosure.

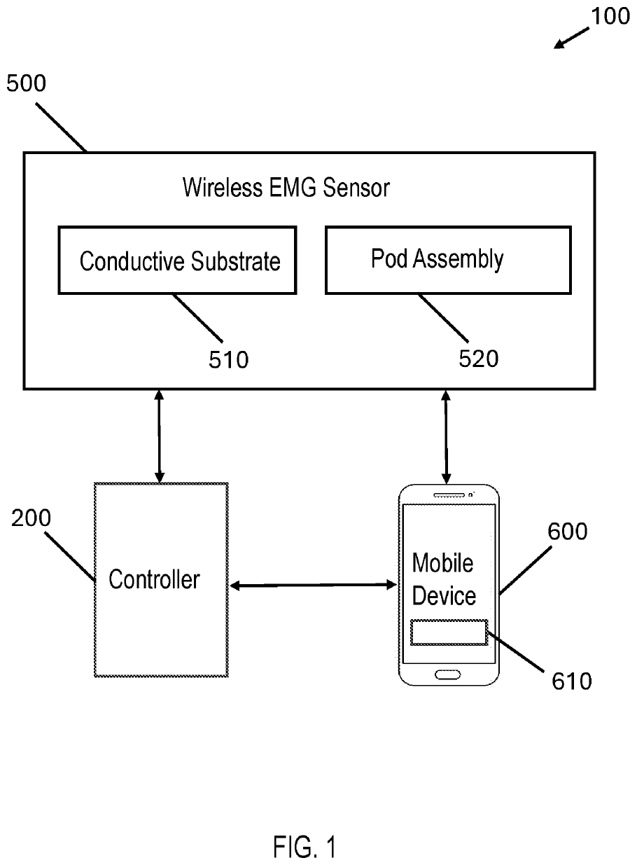

Referring to FIG. 1, there is shown an illustration of an exemplary personal training system 100 in accordance with aspects of the present disclosure. Personal training system 100 is configured to enable efficient and reliable data collection from a user's muscles during exercise. Generally, personal training system 100 collects and provides real-time user exercise feedback in response to the collection and analysis of various user exercise parameters. In doing so, personal training system 100 may compare common muscle activation and intramuscular connectivity patterns in model individuals and patterns in a user to identify training targets, which are used to develop a general training protocol. The configuration of FIG. 1 is exemplary, and variations are contemplated to be within the scope of the present disclosure.

Personal training system 100 includes a controller 200, a wireless electromyography (EMG) sensor 500, and a mobile device 600. Sensor 500 is configured to be adhered in direct contact with the user's dermal muscle surface area and acquire muscle and/or user movement data for analysis (e.g., muscle activation data related to user movement during exercise). Generally, sensor 500 includes a conductive substrate 510 and a pod assembly 520.

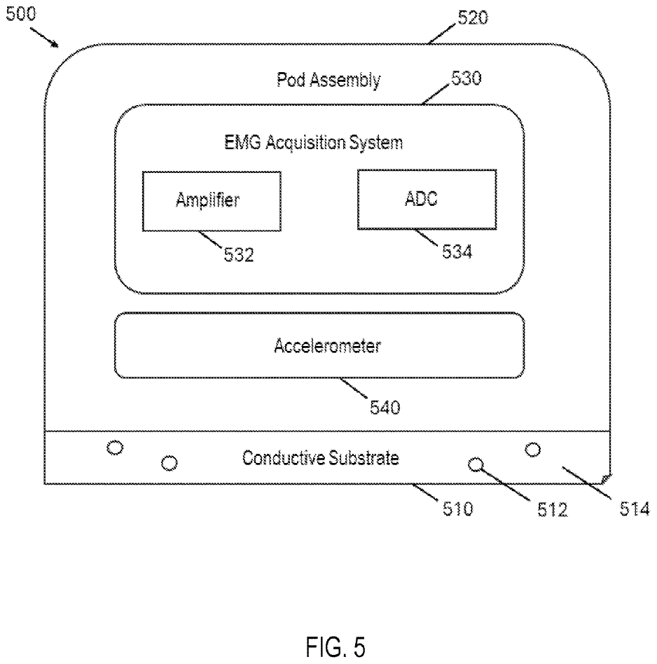

Conductive substrate 510 is configured to detect and measure electrical activity generated by a user's muscles during exercise. To do so, conductive substrate 510 is generally adhered to a user's dermal muscle surface area, such as an exterior dermal surface surrounding a target muscle. Conductive substrate 510 may include a conductive material 512 (FIG. 5), e.g., multi-channel electrodes, which are configured to sense bipolar signals from multiple muscles within a muscle group of the user and provide comprehensive user data to assess a user's exercise form and technique. Conductive substrate 510 may be seamlessly integrated with pod assembly 520 through a wireless connection. In aspects, conductive substrate 510 may be directly connected to pod assembly 520 to create a secure electrical connection, which ensures that pod assembly 520 can transmit data from and/or interact with conductive substrate 510. Conductive substrate 510 may be connected to pod assembly 520 via soldering, conductive adhesives, crimping, wires, magnets, and/or connectors and terminals.

Pod assembly 520 is configured to transmit user data (e.g., EMG data and/or accelerometer data) collected by conductive substrate 510 and accelerometer 540 to an outside device, such as mobile device 600, for data processing. Mobile device 600 may include various type of devices, including a smartphone, tablet, laptop, e-reader, smartwatch, gaming device, virtual reality (VR) headset, and/or other hybrid devices. Pod assembly 520 may transmit user data to multiple user devices 600 in various regions. For example, pod assembly may simultaneously transmit user data to a user's smartphone and laptop via a mobile application.

Generally, pod assembly 520 includes a body made from durable plastics and/or composites such as polytetrafluoroethylene (PTFE), polyethylene (PE), polycarbonate (PC), polysulfone (PSU), polyetheretherketone (PEEK), and/or polyimide (PI), although various materials such as metals, silicone, and/or rubbers and are contemplated and within the scope of the disclosure. In aspects, pod assembly 520 may have waterproof and/or heat resistant, such that pod assembly 520 may be cleaned after exercise using chemical disinfectants and/or soaps (e.g., may be dishwasher-friendly). Pod assembly 520 may include a power source such as a wireless rechargeable battery (not shown).

Various pod assemblies 520 may form a mesh network using Bluetooth Low Energy (BLE), offering low power consumption and compatibility with a wide range of devices including controller 200 and mobile device 600. For example, the mesh network formed by the BLE communication enables seamless data transmission to mobile device 600 to acquire, analyze, and/or output data. In doing so, the mesh network may facilitate communication with an AI model (disclosed below) to effectively assess and provide real-time feedback on a user's exercise form and technique. In aspects, pod assembly 520 may communicate with a controller 200 to manage various functions thereof, such as data output and further analysis, e.g., to transmit collected muscle activation data for processing.

With further reference to FIG. 5, pod assembly 520 may be a compact, wireless device containing various subsystems for acquiring additional user data. Generally, pod assembly 520 includes an EMG acquisition system 530 and an accelerometer 540. EMG acquisition system 530 is configured to obtain muscle activity of a user. In doing so, EMG acquisition system 530 may record electrical signals produced by muscles. Generally, EMG acquisition system 530 includes an amplifier 532 and an analog-to-digital converter (ADC) 534.

Amplifier 532 is configured to increase a signal strength of a recorded electrical signal (e.g., EMG data) to a level suitable for measurement and/or analysis by ADC 534 and may assist in filtering out noise and interference from other electrical sources. In aspects, amplifier 532 may be a low-power instrumentation amplifier, which ensures reliable amplification while maintaining low energy consumption. ADC 534 is configured to convert EMG data via conductive substrate 510 into a digital form for processing. In aspects, ADC 534 may be a small, low-power ADC configured to sense analog EMG data at a frequency of at least 1000 Hz. In aspects, ADC 534 may be configured to digitize the EMG data at a resolution of at least 16 bits.

Accelerometer 540 is configured to monitor a user's movement patterns by measuring acceleration forces of a body part. In doing so, accelerometer 540 may provide a comprehensive view of a user's movement patterns, velocities, and/or orientation data during exercise. In aspects, accelerometer 540 may be a tri-axial accelerometer capable of measuring acceleration along three orthogonal axes, i.e., in three dimensions such as in the x, y, and z axes. Thus, accelerometer 540 provides a benefit over traditional sensors detecting motion by capturing both a magnitude and a direction of acceleration which, when integrated with the EMG acquisition system 530, enables efficient and reliable data collection from various muscles groups and ensures effective assessment of form and technique by an AI model (described below) to provide tailored, real-time feedback. This allows for a more comprehensive understanding of the dynamics of movement associated with muscle activity. For example, accelerometer 540 can detect the orientation of a limb, the speed of movement, and changes in velocity in three-dimensional space. In aspects, accelerometer 540 may perform tasks such as gesture recognition, posture analysis, activity tracking, and/or other tasks where understanding movement patterns is important alongside muscle activity.

Conductive substrate 510 may be implemented in various forms to ensure optimal signal acquisition and user comfort. In aspects, conductive substrate 510 may include sticker-type electrodes, such that sensor 500 is configured as a compact assembly attached to a user's skin. Conductive substrate 510 may be fabricated by screen printing a conductive material 512 onto a flexible base 514 (FIG. 5). The conductive material 512 generally include conductive inks such as silver, carbon, copper, polymer, and/or nanomaterials, which enable the creation of electronic circuits without traditional etching or lithography, allowing for innovative designs and reduced manufacturing costs. The flexible base 514 may include any number of flexible materials, such as polyurethane (PU), polyethylene (PE), polypropylene (PP), composites, elastomers/rubbers, and textiles such as cotton, polyester, nylon, and Kevlar®. Once manufactured, conductive substrate 510 may be covered with an skin-safe adhesive overlay, which ensures a secure bond to a user's skin without causing irritation, discomfort, or adverse reactions (e.g., silicone skin adhesives, transdermal tape, cyanoacrylates). In doing so, various adhesives may be used including acrylic, silicone, hydrocolloid, hydrogel, and pressure-sensitive adhesives (PSAs), which may be hypoallergenic, breathable, moisture resistant, and/or easy to remove without pain or residue.

The sticker-type conductive substrate 510 may be shaped to acquire bipolar signals from various muscles within a muscle group. Thus, sticker-type conductive substrate 510 may be fabricated in several sizes, e.g., small, medium, large, and extra-large, which are designed to accommodate different muscle groups and body areas. A small conductive substrate 510 may be designed for hand use by capturing EMG data from intrinsic hand muscles. A medium conductive substrate 510 may be suitable for forearm and shoulder use by capturing EMG data from muscles such as biceps, triceps, and deltoids. A large conductive substrate 510 may be designed for upper arm, lower leg, and/or hip use by ensuring proper contact with muscles such as the quadriceps, hamstrings, and gluteus maximus. An extra-large conductive substrate 510 may provide for upper leg and back use by allowing for effective data collection from large muscle groups such as the erector spinae and latissimus dorsi.

Moreover, the shape and size of pod assembly 520 may vary based on the placement required. For example, pod assembly 520 may be a small, semicircular-shaped pod configured to adhere to a user's triceps. Conversely, pod assembly 520 may be a long, rectangular-shaped pod configured to adhere to a user's quadriceps. Thus, pod assembly 520 may be strategically placed on a portion of a user's body depending on the exercise(s) to be performed. Where needed, multiple pod assemblies 520 may be aligned to detect specific user motions and/or poses.

In aspects, conductive substrate 510 may be integrated into a garment with woven and/or screen-printed electrodes, such that sensor 500 is configured to be worn by the user in their clothing. This provides an advantage over other similar sensors by offering a convenient and user-friendly alternative to stickers, avoiding the need for additional adhesives and/or attachments. Conductive substrate 510 may be fabricated using conductive fabrics including silver-coated nylon, stainless steel fibers, carbon-based fibers, and/or conductive polymers. The garment-type conductive substrate 510 ensures consistent contact with a user's skin and maintains placement during exercise, allowing for accurate EMG data collection.

Garment-type conductive substrate 510 may be fabricated in the form of any clothing item, such as a hat, shirt, tank top, shorts, pants, and/or stockings. In aspects, garment-type conductive substrate may be integrated with hybrid materials (e.g., hybrid fabrics) to offer unique properties tailored to specific applications, such as moisture wicking, antimicrobial properties, and/or high stretchability.

Pod assembly 520 may be integrated into garment-type conductive substrate 510 and/or provided as a separate device. For example, pod assembly 520 may be sewn into a pocket of a shirt or integrated into the sleeve of a shirt. Alternatively, pod assembly 520 may be a separate device that can be held by a user or attached thereto, e.g., via clothing fasteners, magnets, or other means.

In aspects, conductive substrate 510 may be customized to specific exercises. This provides a benefit over traditional wireless sensors by catering to the unique requirements of an exercise. By focusing on the target muscles involved, personal training system 100 may optimize EMG data acquisition. Customizations for various exercises are detailed below.

For a bench press exercise, the target muscles generally include the pectoralis major and minor, triceps, deltoids, and biceps. Thus, sticker-type conductive substrate 510 can be shaped to cover the chest, upper arm, and/or shoulder regions. Conversely, the garment-type conductive substrate 510 may include conductive material 512 (e.g., threads), which are strategically placed to cover the target muscle groups. Based on the EMG data collected, personal training system 100 may assist the user in maintaining a flat back, avoid excessive arching, and/or engaging their core muscles for stability

For a squat exercise, target muscles generally include the quadriceps, hamstrings, glutes, and core muscles. Therefore, sticker-type conductive substrate 510 may be shaped to cover the upper legs, lower legs, and/or hip regions. The garment-type conductive substrate 510 can incorporate conductive material 512 (e.g., threads) in the target muscle areas, ensuring proper contact with engaged muscles during a squat movement. Based on the EMG data collected, personal training system 100 may assist the user to avoid rounding their lower back, collapsing their chest, and/or allowing their knees to cave inward

For a deadlift exercise, the target muscles typically include the erector spinae, glutes, hamstrings, quadriceps, and core muscles. Thus, sticker-type conductive substrate 510 can be designed to cover the back, hip, and/or leg regions. The garment-type conductive substrate 510 can be woven with conductive material 512 throughout these areas to accurately capture EMG data during the deadlift exercise. Based on the EMG data collected, personal training system 100 may assist the user in maintaining a flat back, engaging their core, and/or keeping the barbell close to their body throughout the deadlift motion.

For an overhead press exercise, the target muscles may include the deltoids, triceps, upper back, and core muscles. Therefore, the sticker-type conductive substrate 510 can be shaped to cover the shoulder and/or upper arm regions, while the garment-type conductive substrate 510 can incorporate conductive material 512 in these areas and across the upper back, ensuring optimal EMG signal acquisition during overhead movement. Based on the EMG data collected, personal training system 100 may assist the user in keeping their back straight, avoiding arching or leaning backward, engaging their core muscles for stability, and/or ensuring that the barbell moves in a straight line overhead, rather than drifting forward or backward.

For a pull-up exercise, the target muscles typically include the latissimus dorsi, teres major, biceps, forearms, shoulder, and core muscles. A sticker-type conductive substrate 510 can be designed to cover the back, shoulder, and/or arm regions. Conversely, a garment-type conductive substrate 510 can incorporate conductive material 512 in these same muscle areas, allowing for effective EMG data collection during the pull-up movement. Based on the EMG data collected, personal training system 100 may assist the user in engaging their core, avoiding rounding their back, and/or over gripping.

For a bent-over row exercise, the target muscles can include the latissimus dorsi, rhomboids, trapezius, biceps, erector spinae, and transverse abdominis muscles. Here, the sticker-type conductive substrate 510 can be shaped to cover the back, shoulder, and arm regions. In the garment-type conductive substrate 510, conductive material 512 may be strategically placed in these same muscle areas to ensure accurate EMG data collection during the bent-over row. Based on the EMG data collected, personal training system 100 may assist the user in keeping their back flat, avoiding rounding or arching their spine, and/or engaging their core muscles for stability

Other exercises and/or sporting activities are contemplated and within the scope of this disclosure. For example, for practicing a golf swing, personal training system 100 may assist the user to achieve the optimal swing and avoid rotating their hips too early. In another example, for marathon training, personal training system 100 may assist the user to decrease their stride length and avoid unnecessary stress on their joints. In still another example, for gymnastics, personal training system 100 may assist the user to maintain proper position and/or posing during a routine, optimizing aerodynamic movements and avoiding injuries.

As will be described in more detail below, the personal training system 100 may implement statistical models and/or machine learning models (e.g., neural network) that process the collected data from sensor 500 to assess a user's exercise form and technique. The term “machine learning model” may include, but is not limited to, neural networks, recurrent neural networks (RNN), generative adversarial networks (GAN), decision trees, Bayesian Regression, Naive Bayes, nearest neighbors, least squares, means, and support vector machine, among other data science and machine learning techniques which persons skilled in the art will recognize.

In aspects, other systems, servers, and/or devices not illustrated in FIG. 1 may be included. In embodiments, one or more of the illustrated components may be omitted. Such and other embodiments are contemplated to be within the scope of the present disclosure.

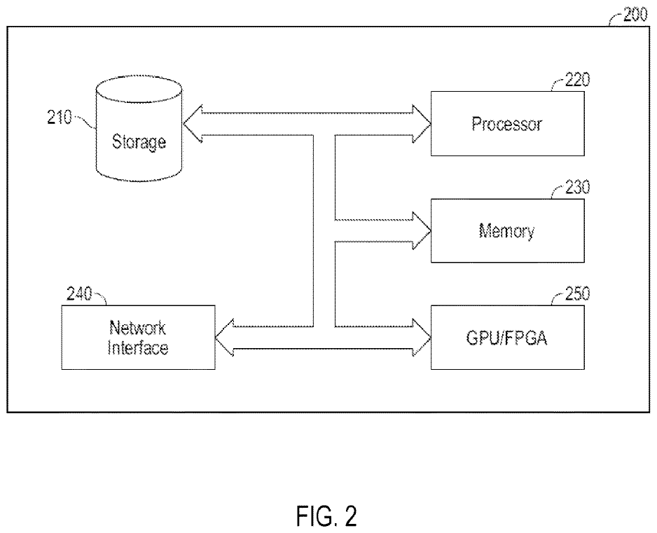

Referring now to FIG. 2, exemplary components of the controller 200 are shown. The controller 200 generally includes a storage or database 210, one or more processors 220, at least one memory 230, and a network interface 240. In aspects, the controller 200 may include a graphical processing unit (GPU) 250, which may be used for processing machine learning network models.

The database 210 can be located in storage. The term “storage” may refer to any device or material from which information may be capable of being accessed, reproduced, and/or held in an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray Disc™, or the like.

In aspects, data may be stored on the controller 200, including, for example, user accounts, workout regimens, statistics, and/or other data. The data can be stored in the database 210 and sent via the system bus to the processor 220. The database 210 may store information in a manner that satisfies information security standards and/or government regulations, such as Systems and Organization Controls (e.g., SOC 2), General Data Protection Regulation (GDPR), and/or International Organization for Standardization (ISO) standards.

As will be described in more detail later herein, the processor 220 executes various processes based on instructions that can be stored in the at least one memory 230 and utilizing the data from the database 210. With reference also to FIG. 1, a request from a user device, such as mobile device 600 or a client computer, can be communicated to the controller 200 through a network interface. The illustration of FIG. 2 is exemplary, and persons skilled in the art will understand that other components may exist in controller 200. Such other components are not illustrated for clarity of illustration.

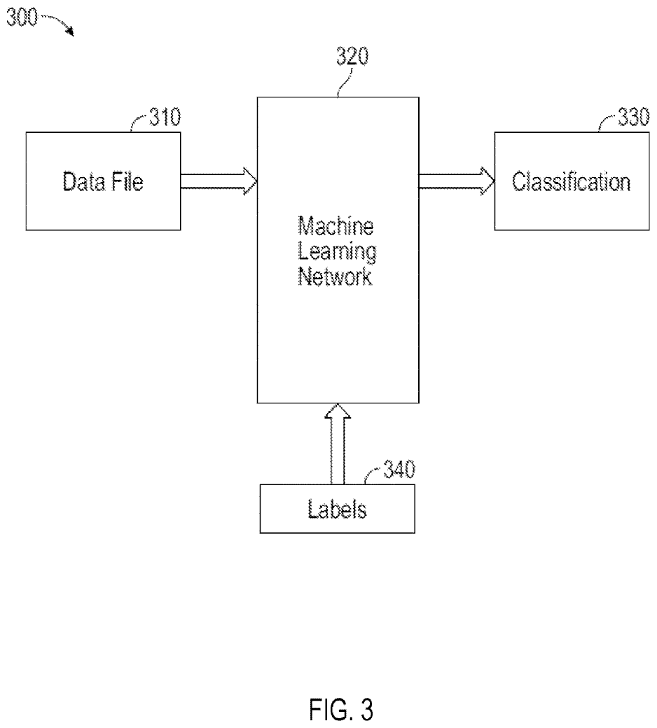

With reference to FIG. 3, a block diagram for a machine learning network 320 for classifying data in accordance with some aspects of the disclosure is shown. In some systems, a machine learning network 320 may include, for example, a convolutional neural network (CNN), a regression and/or a recurrent neural network. A deep learning neural network includes multiple hidden layers. As explained in more detail below, the machine learning network 320 may leverage one or more classification models 330 (e.g., CNNs, decision trees, a regression, Naive Bayes, k-nearest neighbor) to classify data. In aspects, the classification model 300 may use a data file 310 and labels 340 for classification. The machine learning network 320 may be executed on the controller 200 (FIG. 2). Persons of ordinary skill in the art will understand the machine learning network 320 and how to implement it.

In machine learning, a CNN is a class of artificial neural network (ANN). The convolutional aspect of a CNN relates to applying matrix processing operations to localized portions of data, and the results of those operations (which can involve dozens of different parallel and serial calculations) are sets of many features that are delivered to the next layer. A CNN typically includes convolution layers, activation function layers, deconvolution layers (e.g., in segmentation networks), and/or pooling (typically max pooling) layers to reduce dimensionality without losing too many features. Additional information may be included in the operations that generate these features. Providing unique information, which yields features that give the neural networks information, can be used to provide an aggregate way to differentiate between different data input to the neural networks.

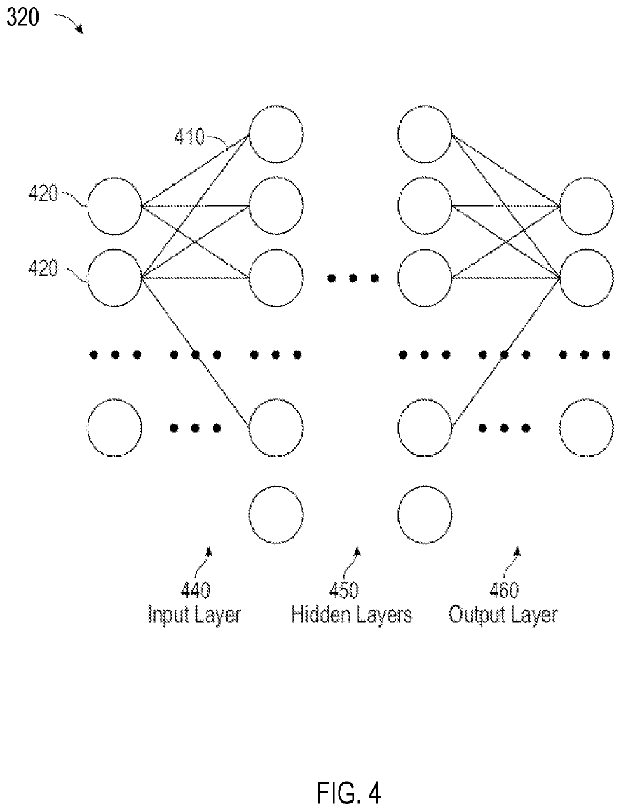

Referring to FIG. 4, generally, a machine learning network 320 (e.g., a convolutional deep learning neural network) includes at least one input layer 440, a plurality of hidden layers 450, and at least one output layer 460. The input layer 440, the plurality of hidden layers 450, and the output layer 460 all include neurons 420 (e.g., nodes). The neurons 420 between the various layers are interconnected via weights 410. Each neuron 420 in the machine learning network 320 computes an output value by applying a specific function to the input values coming from the previous layer. The function that is applied to the input values is determined by a vector of weights 410 and a bias. Learning, in the deep learning neural network, progresses by making iterative adjustments to these biases and weights. The vector of weights 410 and the bias are called filters (e.g., kernels) and represent particular features of the input (e.g., a particular shape). The machine learning network 320 may output logits. Although CNNs are used as an example, other machine learning classifiers are contemplated.

The machine learning network 320 may be trained based on labeling training data to optimize weights. For example, samples of feature data may be taken and labeled using other feature data. In some methods in accordance with this disclosure, the training may include supervised learning or semi-supervised. Persons of ordinary skill in the art will understand training the machine learning network 320 and how to implement it.

In aspects, machine learning network 320 may include a machine learning platform such as TensorFlow®, PyTorch®, or a similar platform. Machine learning network 320 may be configured to learn a generalized model of proper actions performed by individuals, such as athletes and professionals, by analyzing the relationships and/or patterns in extracted features from a dataset. As previously discussed, machine learning network 320 may be trained using techniques such as supervised and unsupervised learning, which enable prediction of a proper form and/or action from labeled examples within the dataset. For example, machine learning network 320 may use supervised learning to identify common muscle activation and/or intermuscular connectivity patterns from a dataset including EMG signals from highly trained athletes. In another example, machine learning model 320 may use unsupervised learning to classify common muscle activation and/or intermuscular connectivity patterns from a user (e.g., trainee). Machine learning network 320 may compare common muscle activation and/or intermuscular connectivity patterns in athletes and users to identify training targets and develop a general training protocol. The training process involves optimizing the model's parameters to minimize the error in its predictions, using techniques like backpropagation and gradient descent.

For example, machine learning network 320 may be trained through EMG and/or accelerometer data collected during exercise from a diverse range of highly trained individuals, such as athletes and human performance professional across various sports and weightlifting disciplines. In doing so, individuals may be selected to represent different age groups, skill levels, and/or body types. The collected EMG and/or accelerometer data may be preprocessed to ensure focus on relevant features and/or patterns in the data, leading to more accurate and reliable feedback. For example, preprocessing may remove noise and artifacts, normalize data, and/or segment data. Data pre-processing may be performed by various modules including the EMG acquisition system 530 and/or mobile device 600. For example, data may be segmented into meaningful partitions using information from the accelerometer and/or EMG. A band-pass filter may be applied to the raw EMG signals within the 20-500 Hz frequency range to remove unwanted noise.

In aspects, an AI model may be configured to extract relevant features from preprocessed EMG and/or accelerometer data using advanced neural network architectures, such as graph neural networks (GNNs). This provides an advantage over traditional neural networks by more effectively exploiting the spatial and temporal relationships between muscles. For example, machine learning network may extract features such as an amplitude of EMG, an amplitude distribution across muscles of EMG, inter-muscular coherence, inter-muscular connectivity, and/or weighted phase lag coherence between muscles of EMG, fused with accelerometer data, as described below.

Inter-muscular coherence relates to the degree to which electrical signals from different muscles are synchronized and/or coherent with one other (e.g., in terms of frequency and phase) during a coordinated movement or activity. Inter-muscular coherence often occurs at specific frequency bands corresponding to the oscillatory rhythms of neural activity involved in motor control, which may vary depending on the task and the muscles involved. Using personal training system 100, inter-muscular coherence is computed to assess the degree of synchronization between a user's muscle pairs. Here, coherence is calculated as:

$$[ {C}_{x y} ( f ) = \frac{{\vert {P}_{x y} ( f ) \vert}^{2}}{{P}_{x x} ( f ) {P}_{y y} ( f )} ]$$

C_{xy}(f) represents the coherence between two EMG signals x and y at frequency f, Pₓₓ(f) and P_{yy}(f) are the power spectral densities of the signals, and P_{xy}(f) is the cross power spectral density.

Intra-muscular connectivity relates to the communication and coordination between different parts and/or regions within a single muscle, e.g., individual muscle fibers and/or cells. Different regions within a muscle may have specialized functions or contribute differently to muscle function. Thus, intra-muscular connectivity may involve the segregation of muscle fibers into functional compartments or the coordination of specific muscle fiber types for different tasks. Using personal training system 100, intra-muscular connectivity is computed to assess connections and/or interactions within a muscle. Non-negative matrix factorization (NMF) is used to analyze the intra-muscular connectivity patterns in the EMG data. The raw EMG signal matrix, X, is factorized into two non-negative matrices W and H, as follows:

[X≈WH]

W represents the spatial activation patterns, and H represents the temporal activation patterns of the EMG signals. The connectivity patterns within a single muscle are then assessed by analyzing these spatial and temporal activation patterns.

Weighted phase lag coherence relates to the level of synchronization between pairs of muscles, e.g., via the phase difference between two signals at each time point calculated, while minimizing the influence of volume conduction effects. This measure is computed using the following equation:

$$[ W P L {C}_{x y} ( f ) = \frac{\vert Im [ {S}_{x y} ( f ) ] \vert}{\sqrt{{P}_{x x} ( f ) {P}_{y y} ( f )}} ]$$

WPLC_{xy}(f) denotes the weighted phase lag coherence between EMG signals x and y at frequency f, Pₓₓ(f) and P_{yy}(f) represent the power spectral densities, and Im[S_{xy}(f)] is the imaginary part of the cross-spectrum between the signals.

After extracting these features, feature selection techniques such as mutual information, principal component analysis (PCA), and/or recursive feature elimination (RFE) are applied to identify the most important features for accurately representing proper form and technique. The selected features are then utilized to train and validate machine learning network 320, ensuring optimal performance in providing feedback.

In addition, Intra-Muscular Coherence (IMC) is introduced to form a network of spectral information from target muscles across various frequency ranges, offering a unique view of muscle co-activation. IMC is the level of correlation between two muscles in the frequency domain, indicating the common neural drive. IMC's functionality spans across different frequency bands implicated in aspects of neuromuscular control and neuromotor disease. This utilization of spectral information enables the personal training system 100 to provide improved feedback during exercise, adapted to individual needs and conditions. IMC functions across various frequency bands, each implicated in different aspects of neuromuscular control and neuromotor disease. Thus, user exercise feedback may be adjusted in response to altered neural drives under conditions of neuromotor disease or induced muscle pain. Alpha-band neural drive (α: 8-12 Hz) is associated with inter-muscular coordination of several muscles. Beta-band neural drive (β: 13-30 Hz) relates to descending cortical control of isometric muscle movement and is dependent on the health of the cortical-spinal tract. Gamma-band neural drive (γ: >30 Hz) to muscles has been identified in dynamic movements as studied with IMC. Coordinated muscle activity is dependent on the neural drive itself, making IMC an ideal mechanism for assessing the neural drive to muscles.

Once trained, machine learning network 320 is configured to analyze to analyze a user's real-time EMG and accelerometer data to determine the feedback, including a proper form and/or action (e.g., technique). In doing so, machine learning network 320 compares the user's current performance with a generalized model of proper form and/or actions derived from the dataset. Machine learning network 320 uses this analysis to generate real-time feedback for the user.

Generally, the real-time feedback is generated and/or presented on mobile device 600. The feedback may be presented within a software application on mobile device 600, such as mobile application 610 (described below), which guides the user to adjust their physical activities and/or weightlifting exercises. The feedback presented by mobile device 600 may reduce the risk of injury and enhances the user's overall performance by ensuring the user utilizes the proper form and/or action. For example, mobile device 600 may direct a user to avoid overarching their back and/or bending their knees based on a current exercise, EMG and accelerometer data.

Mobile device 600 may include mobile application 610, which pairs with sensor 500 via a BLE chip, ensuring efficient and real-time reception of EMG and/or accelerometer data. Mobile application 610 is configured to process and display the EMG and/or accelerometer data with the real-time feedback generated by machine learning network 320 in an intuitive format. In doing so, mobile application 610 guides the user to adjust their form and/or actions during physical activities, thereby enhancing user performance and reducing the risk of injury. In aspects, machine learning network 320 may enable a user to develop user-specific training targets and/or training protocol using the user's unique muscle activation and/or intermuscular connectivity patterns.

Mobile application 610 may be configured to display the analyzed information from machine learning network 320, to provide the real-time feedback to the user to adjust their movements. In aspects, mobile application 610 is configured to integrate with commonplace fitness applications (e.g., MyFitnessPal®, Fitbit®, Peloton®, and/or MapMyFitness®), allowing the user to consolidate exercise data and seamlessly track their fitness progress and/or goals. For example, mobile application 610 may extract profile data from other applications and/or transit data thereto based on user-entered data.

In aspects, mobile application 610 may include a user-friendly interface, which is easily navigable and enables user access to key features. For example, a user can customize their profiles with physiological and/or demographic data, such as body height, weight, gender, age, and/or ethnicity. In aspects, a user may upload an image of their face and/or body to mobile application 610, which enables calculation of limb and/or torso lengths for more accurate exercise guidance. Mobile application 610 may include progress tracking and goal setting features, which enable a user to monitor their progress and stay motivated throughout their fitness journey.

The term “application” may include a computer program and/or machine-readable instructions designed to perform particular functions, tasks, or activities for the benefit of a user. Application may refer to, for example, software running locally or remotely, as a standalone program or in a web browser, or other software that would be understood by one skilled in the art to be an application. An application may run on controller 200, a server, or on a user device, including, for example, on mobile phone 600.

In aspects, the real-time feedback generated by machine learning network 320 may include haptic, visual and/or auditory cues, which may be transmitted to mobile device 600 to alert the user via mobile application 610. For example, mobile application 610 may produce a scaled vibration, which increases or decreases in intensity and/or speed based on the proximity of a user's position to an ideal position. In aspects, haptic (e.g., tactile) feedback may include specific vibration patterns based on the exercise and/or feedback. In another example, mobile application 610 may display a visual alert, including a graphical or text representation of a recommended form and/or action. In aspects, mobile application 610 may display graphical representations of the user's performance overlaid with an ideal form and/or action and provide specific recommendations for improvement, and/or a textual representation of the recommendation (e.g., move right leg forward 2-3 inches).

In another example, mobile application 610 may produce an auditory alert. The auditory alert can include a general sound such as beep, alarm and/or song based on a trigger (e.g., leg out of proper position), and/or produce a verbal recommendation (e.g., “pick up elbows”). In aspects, machine learning network 320 may use natural language processing (NLP) to relay auditory cues to a user through mobile application 610. While the cues are described as being produced by mobile device 600 and/or mobile application 610, it is contemplated that the haptic, visual and/or auditory cues may be performed by a number of other devices, such as pod assembly 130 (e.g., Arduino® within the pod emits a sound). Moreover, any of the haptic, visual and/or auditory cues may be pre-programmed and/or customized by a user.

In aspects, user session data (e.g., user exercise data) may be saved for future referencing. For example, a user may save session data within their profile, which may be accessed at a later date. Various data, including workouts, muscles, activated, workout feedback, and/or graphical images may be saved in this manner. Data may be saved via a network, controller 200, and/or directly on mobile device 600.

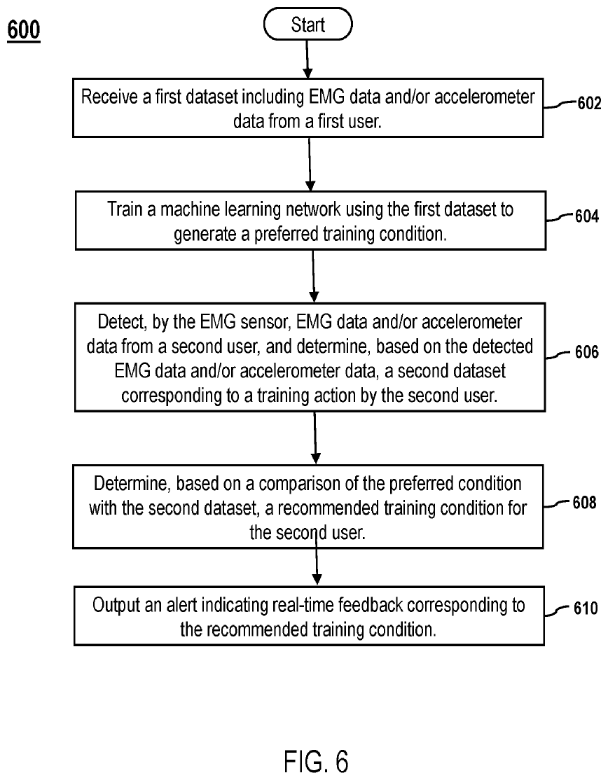

FIG. 6 shows a method 600 for an exemplary use of personal training system 100. Although the steps of method 600 of FIG. 6 are shown in a particular order, the steps need not all be performed in the specified order, and certain steps can be performed in another order. In various aspects, the method 600 of FIG. 6 may be performed all or in part by controller 200 of FIG. 1. In other aspects, the method 600 of FIG. 6 may be performed all or in part by another device, for example, a mobile device and/or a client computer system. These and other variations are contemplated to be within the scope of the present disclosure.

Initially, at step 602, the controller 200 causes system 100 to receive a first dataset including EMG data and/or accelerometer data from a first user. Generally, the first user is one or more individuals selected from a diverse range of highly trained individuals, such as athletes and human performance professionals across various sports and weightlifting disciplines. For example, to generate a first dataset for determining an ideal squatting pose, the first user may be a group of professional weightlifters. Conversely, to generate a first dataset for determining an ideal stride length, the first user may be a group of professional marathon runners or trainers.

Next, at step 604, the controller 200 causes system 100 to train a machine learning network using the first dataset to generate a preferred training condition. In training the machine learning network, various supervised and unsupervised learning techniques may be used, which enable prediction of a proper form and/or action from labeled examples within a second dataset. For example, machine learning network 320 may use supervised learning to identify common muscle activation and/or intermuscular connectivity patterns from a dataset including EMG signals from highly trained athletes. In aspects, machine learning model 320 may also use unsupervised learning to classify common muscle activation and/or intermuscular connectivity patterns from a user. The generated preferred training condition is generally an ideal form and/or action (e.g., technique) determined from analysis of the professional first user(s) while exercising, which indicates an ideal form for exercising.

Next, at step 606, the controller 200 causes system 100 to detect EMG data and/or accelerometer data from the second user. For example, a user may begin engaging in physical exercise (e.g., perform a squat, pullup, and/or deadlift) with sensor 500 adhered to their dermal muscle surface area, which enables detecting of muscle activity such as muscle contraction and subsequent body movements. Based on the detected EMG data and/or accelerometer data, a second dataset may be determined corresponding to a training action by the second user. For example, sensor 500 may detect EMG data from EMS acquisition system 530 and accelerometer data from accelerometer 540.

The EMG data and/or accelerometer data may be amplified by amplifier 532 to increase a signal strength of the EMG data to a level suitable for measurement and/or analysis and may assist in filtering out noise and interference from other electrical sources. The accelerometer data may be digitized by ADC 534 for subsequent processing by system 100. For example, ADC may digitize the EMG data at a resolution of at least 16 bits. Once ready for processing, the EMG data and/or accelerometer data is sent to mobile device 600 and/or controller 200. Generally, the data is transmitted via BLE connectivity.

Next, at step 608, the controller 200 causes the system 100 to determine a recommended training condition for the second user based on a comparison of the preferred condition with the second dataset. Generally, the recommended condition is a recommended form and/or action (e.g., technique) for the user to perform, which may prevent injury and/or further a fitness goal. For example, the system 100 may determine that a user is buckling their knees during weightlifting and determine an alternative form.

The comparison and/or analysis above may be performed by via machine learning network 320 to predict the user's performance in terms of proper form and technique. This provides the benefit of generating tailored user feedback and/or guidance, as opposed to traditional personal training systems, which merely provide charted raw sensor data. For example, in response to an improper barbell squat, machine learning network 320 may note discrepancies in the alpha-band neural drive associated with inter-muscular coordination between the user's quadriceps and gluteal muscles. In another example, during a deadlift, machine learning network 320 may recognize a reduced beta-band neural drive associated with the control of isometric muscle movement in the user's core muscles compared to the professional baseline (e.g., the first dataset). Thus, personal training system 100 provides the benefit of analyzing the user's form and technique and providing a response in real-time.

The recommended training condition may be related to various exercises, such as bench pressing, squatting, deadlifting, overhead pressing, pull-ups, and/or bent-over rows. In aspects, a custom and/or specific workout plan may be entered by a user to determine a tailored recommended training condition. Other exercises and/or sporting activities are contemplated and within the scope of this disclosure. For example, the recommended training condition may be related to a stride length for marathon trainer or a swing and/or hip rotation for a golf trainer.

Next, at step 610, the controller 200 causes the system 100 to output an alert indicating real-time feedback corresponding to the recommended training condition. The real-time feedback generated by machine learning network 320 may include haptic, visual and/or auditory cues, which may be transmitted to mobile device 600 to alert the user via mobile application 610. For example, if a user is close to arching their back, mobile device 600 may emit a strong vibration to alert the user and prevent further back injury.

Further, mobile application 610 may display graphical representations of the user's performance overlaid with an ideal form and/or technique, while providing specific recommendations for improvement. For example, in response to a barbell squat, mobile application 610 may deliver feedback directing the user to push their knees outward, thereby improving their form and reducing the risk of injury. In another example, in response to a deadlift, application 610 may deliver feedback directing the user to engage their core and maintain a neutral spine to foster appropriate form and minimize injury risk.

While described for use in personal training, it will be understood that system 100 may be implemented for use in various settings. For example, system 100 may be used in the medical field, sporting fields, and/or recreational fields.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages that are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above are also intended to be within the scope of the disclosure.

What is claimed is:

1. A system for personal training, comprising:
a sensor configured to detect at least one of EMG data or accelerometer data;
a processor; and
a memory coupled to the processor, the memory having instructions stored thereon, which when executed by the processor, cause the system to:
receive a first dataset including at least one of EMG data or accelerometer data from a first user;
train a machine learning network using the first dataset to generate a preferred training condition;
detect, by the sensor, at least one of EMG data or accelerometer data from a second user;
determine, based on the detected at least one of EMG data or accelerometer data from the second user, a second dataset corresponding to a training action by the second user; and
determine, based on a comparison of the preferred training condition with the second dataset, a recommended training condition for the second user.

2. The system of claim 1, wherein the sensor includes a pod assembly and a conductive substrate.

3. The system of claim 2, wherein the pod assembly includes at least one of an amplifier, an accelerometer, or an analog-to-digital (ADC) converter.

4. The system of claim 3, wherein the accelerometer is tri-axial accelerometer configured to detect acceleration along three axes.

5. The system of claim 2, wherein the conductive substrate includes a conductive sticker configured to attach to the second user's skin.

6. The system of claim 2, wherein the conductive substrate includes a conductive fabric configured to be woven into a garment worn by the second user.

7. The system of claim 1, wherein the instructions, when executed by the processor, further cause the system to:
output an alert indicating real-time feedback corresponding to the recommended training condition,
wherein the alert includes at least one of a haptic, visual, or auditory cue.

8. The system of claim 1, wherein the instructions, when executed by the processor, further cause the system to:
analyze, by the machine learning network, the second dataset to determine a plurality of features corresponding to the training action by the second user, wherein the plurality of features includes at least one of an EMG amplitude, an amplitude distribution, an inter-muscular coherence, an intra-muscular connectivity, or a weighted phase lag coherence; and
preprocess the at least one of EMG data or accelerometer data from the second user prior to determining the second dataset, wherein preprocessing the at least one of EMG data or accelerometer data includes at least one of removing noise, normalizing data, or segmenting data.

9. The system of claim 1, wherein the recommended training condition corresponds to at least one of a medical application, a sports application, or a recreational application.

10. The system of claim 1, wherein a plurality of sensors is communicatively coupled using Bluetooth low energy (BLE) to form a network.

11. A processor-implemented method for personal training, the method comprising:
receiving a first dataset including at least one of EMG data or accelerometer data from a first user;
training a machine learning network using the first dataset to generate a preferred training condition;
detecting, by a sensor, at least one of EMG data or accelerometer data from a second user;
determining, based on the detected at least one of EMG data or accelerometer data from the second user, a second dataset corresponding to a training action by the second user; and
determining, based on a comparison of the preferred training condition with the second dataset, a recommended training condition for the second user.

12. The method of claim 11, wherein the sensor includes a pod assembly and a conductive substrate.

13. The method of claim 12, wherein the pod assembly includes at least one of an amplifier, an accelerometer, or an analog-to-digital (ADC) converter.

14. The method of claim 13, wherein the accelerometer is tri-axial accelerometer configured to detect acceleration along three axes.

15. The method of claim 12, wherein the conductive substrate includes a conductive sticker configured to attach to the second user's skin.

16. The method of claim 12, wherein the conductive substrate includes a conductive fabric configured to be woven into a garment worn by the second user.

17. The method of claim 11, wherein the method further comprises:
outputting an alert indicating real-time feedback corresponding to the recommended training condition,
where the alert includes at least one of a haptic, visual, or auditory cue.

18. The method of claim 11, wherein the method further comprises:
analyzing, by the machine learning network, the second dataset to determine a plurality of features corresponding to the action by the second user, wherein the plurality of features includes at least one of an EMG amplitude, an amplitude distribution, an inter-muscular coherence, an intra-muscular connectivity, or a weighted phase lag coherence; and
preprocessing the at least one of EMG data or accelerometer data from the second user prior to determining the second dataset, wherein preprocessing the at least one of EMG data or accelerometer data includes at least one of removing noise, normalizing data, or segmenting data.

19. The method of claim 11, wherein the recommended training condition corresponds to at least one of a medical application, a sports application, or a recreational application.

20. A non-transitory computer readable storage medium including instructions that, when executed by a computer, cause the computer to perform a method for personal training, the method comprising:
receiving a first dataset including at least one of EMG data or accelerometer data from a first user;
training a machine learning network using the first dataset to generate a preferred training condition;
detecting, by a sensor, at least one of EMG data or accelerometer data from a second user;
determining, based on the detected at least one of EMG data or accelerometer
data from the second user, a second dataset corresponding to a training action by the second user; and
determining, based on a comparison of the preferred training condition with the second dataset, a recommended training condition for the second user.
